(19) United States
(12) Patent Application Publication
(10) Pub. No.: US 2016/0166794 A1
(43) Pub. Date: Jun. 16, 2016

(54) Swivel Connector

(71) Applicant: MERCURY ENTERPRISES, INC., Clearwater, FL (US)

(72) Inventors: Jeffrey B. Ratner, Pinellas Park, FL (US); James Russell Ritter, III, Largo, FL (US)

(73) Assignee: MERCURY ENTERPRISES, INC., Clearwater, FL (US)

(21) Appl. No.: 14/566,303

(22) Filed: Dec. 10, 2014

(51) Int. Cl. A61M 16/08; A61M 16/20; A61M 16/06

(52) U.S. Cl. CPC A61M 16/0816; A61M 16/06; A61M 16/208

(57) ABSTRACT

A swivel connector includes a patient member that has a first end for connecting to a patient facemask. The first end of the patient member is in fluid communication with a second end of the patient member. A first end of a gas supply member is for connecting to a supply of gases. A second end of the gas supply member is rotatably and fluidly coupled to the second end of the patient member, thereby gases flow between the patient member and the gas supply member. An axis of the first end of the patient member is at a non-zero angle with respect to an axis of the second end of both members. Likewise, an axis of the first end of the gas supply member is at a non-zero angle with respect to an axis of the second end of both members.

### FIELD

This invention relates to the field of medical devices and more particularly to a system for providing angular attachment of a source of gases to a patient mask.

### BACKGROUND

When administering gases (e.g. oxygen, etc.) to a patient, often the patient is fitted with a facemask that covers the patient's nose and/or mouth. In many situations, such a facemask is worn by the patient for extended periods of time, often for the entire day. Depending upon the elevation of the patient, the angle of the tube that conveys the gases is often compensated by a connector that is fitted onto the facemask, in between the facemask and the tube that delivers the gases.

In some patient elevations, such as when sitting up, it is desired to have the gas tube extend directly outward from the patient, somewhat perpendicular to the general shape/plane of the facemask. In other patient elevations, it is desired to have the gas tube extend outward at an angle, often at a right angle to the above perpendicular direction, allowing the tube to be routed to the source of gases with less potential of bending and kinking.

In the past, to provide such variations in direction of the tube exiting the facemask, multiple fittings or connectors were made available. For example, for a sitting patient, a straight connector is removably attached (in some examples) to the facemask, extending outwardly in a generally perpendicular direction with respect to the overall shape of the facemask. Later, when the patient is reclined into the horizontal position for sleeping, the straight connector is removed and an elbow connector is removably attached (in some examples) to the facemask, extending outwardly in a generally parallel direction with respect to the overall shape of the facemask.

The opposite is performed when the patient is later moved from the horizontal position to a more vertical position. Having the gas tube in the wrong orientation creates discomfort for the patient and a fixed interface creates a burden on the clinician.

Further, in the past, many medical facilities purchased two facemasks, one with each orientation of the gas tube, requiring a change-out of the entire facemask to change orientation of the gas tube, as is often needed when transitioning from, say an ambulance to a hospital room, etc. This leads to unnecessary costs, environmental waste, and extra devices that need to be sterilized.

The above mentioned solutions have worked in the past, but require time and labor to remove one fitting from the facemask and then disconnect the gas tube, then install a different fitting onto the facemask and then reconnect the gas tube to the second fitting.

Changing of either the facemask or a fitting in the gas supply not only results in increased cost, work, and frustration, but additionally, during such changing, the therapy being provided to the patient is interrupted and allows contamination to enter the system.

What is needed is a device that will remain in the airway circuit, yet allow redirection of the gas tube with respect to the facemask.

### SUMMARY

In one embodiment, a swivel connector is disclosed including a patient member that has a first end for connecting to a patient facemask and has a second end. The first end of the patient member is in fluid communication with the second end of the patient member. A gas supply member has a first end for connecting to a supply of gases and has a second end that is rotatably coupled and fluidly coupled to the second end of the patient member, thereby the patient member is rotatable with respect to the gas supply member and gases flow between the patient member and the gas supply member. An axis of the first end of the patient member is at a non-zero angle with respect to an axis of the second end of the patient member. An axis of the first end of the gas supply member is at a non-zero angle with respect to an axis of the second end of the gas supply member. The axis of the second end of the patient member is in alignment with the axis of the second end of the gas supply member.

In another embodiment, a method of adjusting an angle of a connection between a supply of gases and a facemask is disclosed including connecting a first end of a patient member to a gas supply port of the facemask. The patient member having a second end and the first end of the patient member is in fluid communication with the second end of the patient member. A first end of the gas supply member is connected to the supply of gases. The gas supply member has a second end that is rotatably coupled and fluidly coupled to the second end of the patient member, thereby the patient member is rotatable with respect to the gas supply member and gases flow between the patient member and the gas supply member. The axis of the first end of the patient member is at a 45 degree angle with respect to the axis of the second end of the patient member and the axis of the first end of the gas supply member is at a 45 degree angle with respect to the axis of the second end of the gas supply member, thereby enabling the axis of the first end of the patient member to be adjusted to any angle between zero degrees and 90 degrees with respect to the axis of the first end of the gas supply member. The method continues with rotating the patient member with respect to the gas supply member, therefore adjusting the angle between the axis of the first end of the patient member and the axis of the first end of the gas supply member from zero degrees to 90 degrees.

In another embodiment, a system for providing gases from a tube to a patient is disclosed including a facemask for interfacing with an airway of the patient with a patient member that has a first end connected to a port on the facemask and has a second end. The first end is in fluid communication with the second end enabling flow of gases through the patient member. A gas supply member has a first end for connecting to the tube (gas supply) and has a second end that is rotatably coupled and fluidly coupled to the second end of the patient member, thereby the patient member is rotatable with respect to the gas supply member and gases flow between the first end of the patient member and the first end of the gas supply member. An axis of the first end of the patient member is at a non-zero angle with respect to an axis of the second end of the patient member. Furthermore, an axis of the first end of the gas supply member is at a non-zero angle with respect to an axis of the second end of the gas supply member and the axis of the second end of the patient member is in alignment with the axis of the second end of the gas supply member. Therefore, through rotation of the gas supply member with respect to the patient member, the angle between the axis of the first end of the gas supply member is adjustable with respect to the axis of the first end of the patient member.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates an exploded view of a swivel connector in conjunction with a facemask.

FIG. 2 illustrates an exploded view of the swivel connector.

FIG. 3 illustrates an elevational view of the swivel connector.

FIG. 4 illustrates a sectional view of the swivel connector along lines 4-4 of FIG. 3.

FIG. 5A illustrates a plan view of the swivel connector in a perpendicular configuration.

FIG. 5B illustrates a plan view of the swivel connector in an elbow configuration.

FIG. 6 illustrates a perspective view of a flapper valve of the swivel connector.

FIG. 7 illustrates a perspective view of the patient airway side of the swivel connector.

FIG. 8 illustrates a perspective view of a valve retainer of the swivel connector.

FIG. 9 illustrates a perspective view of the gas supply side of the swivel connector.

FIG. 10 illustrates a perspective view of a retaining ring of the swivel connector.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

Throughout the following, a specific embodiment of a swivel connector 10 is shown that accomplishes all of the intended functions. Although the swivel connector 10 described and shown in the figures has been tested and is known to work very well, other embodiments providing the same or similar functionality are equally anticipated and there are no limitations to the disclosed invention inherited from the exemplary design presented.

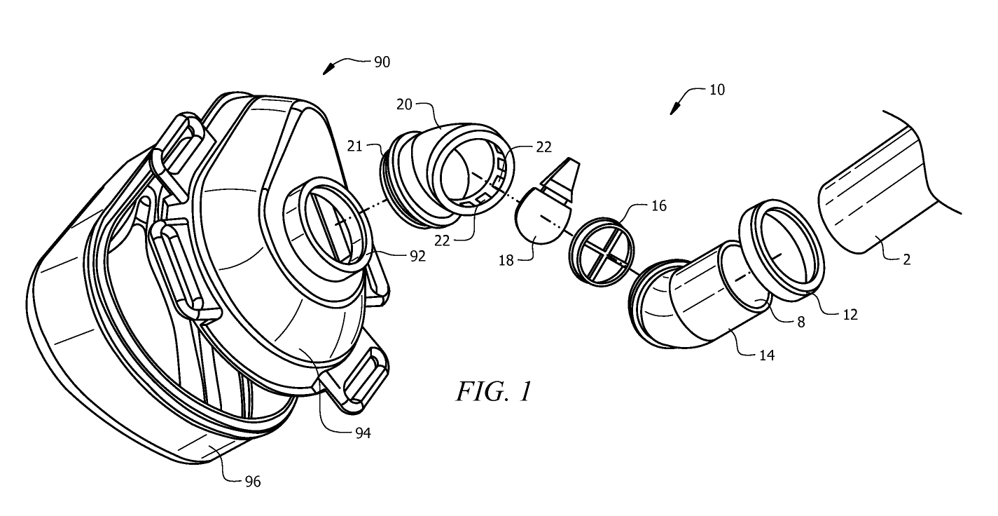

Referring to FIG. 1, an exploded view of a swivel connector 10 in conjunction with a facemask 90 is shown. The facemask 90 is shown for completeness and has two pieces, a gas supply interface 92/94 and a face interface 96. A gas supply port 92 receives gas (e.g., oxygen, fresh air, nebulized medication, etc.) from a gas supply (through the swivel connector 10) and fills the cavity formed by the cover member 94 and the face interface 96 with the gas. In some such facemasks 90, the cover member 94 and the face interface 96 are removable from each other for cleaning, etc. The face interface 96 has an opening through which the gas is delivered to the patient through the patient's nose and/or mouth.

Also shown in FIG. 1 is a gas supply tube end 2, which is the termination of a longer tube for supplying gas to the patient, as known in the medical industry. In the past, the gas supply tube end was connected directly to the facemask 90 or connected to the facemask by a fixed connector, either a straight fixed connector that extended somewhat perpendicular to the plane of the face of the patient wearing the facemask 90 or an elbow connector that made a 90 degree turn, thereby extending somewhat parallel to this plane. Having two connectors, the clinician attaches a straight fixed connector for some circumstances, then removes the straight fixed connector and attaches an elbow connector for other circumstances, etc. This requires an interruption of therapy during the removal/attachment of the connector and, having multiple connectors often leads to one or the other connectors getting lost.

The individual components of the swivel connector 10 are shown in relationship to each other. The patient member 20 has, in this example, a snap interface 21 that snaps into the gas supply port 92 of the facemask 90 and the gas supply member 14 has a port 8 that connects to the gas supply tube end 2. The patient member 20 is rotatably held to the gas supply member 14 by a snap-on collar 12. A flapper valve assembly 16/18 is optionally provided to prevent asphyxiation of the patient in the event that the source of gas abates (e.g. CPAP failure).

Although not required, but preferred, detents 22 are formed around a surface of either the patient member 20 or the gas supply member 14 to hold the patient member 20 or the gas supply member 14 in a particular position of rotation with respect to each other after being positioned as desired, otherwise, the gas supply member 14 would rotate freely with respect to the patient member 20, dependent upon friction between the such.

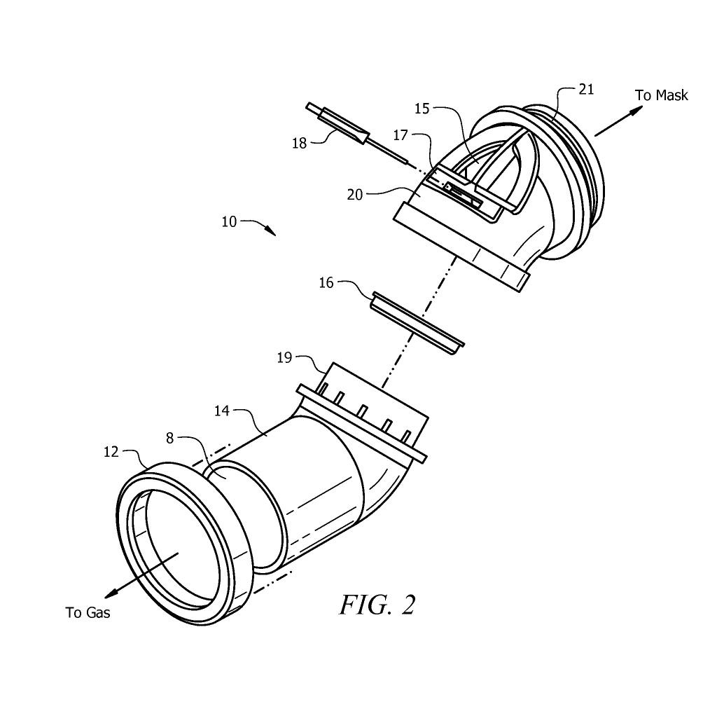

Referring to FIG. 2, an exploded view of the swivel connector 10 is shown. In this, the relationship of the flapper valve 16/18 is understood. The flapper valve 16/18 has a flapper screen 16 that is positioned in the air flow up stream (towards the gas supply) from the flapper 18 and the flapper 18 is inserted into the patient member 20 through a slit 17. As gas flows from the gas supply member 14 towards the mask connection 21, the flapper 18 deflects and covers the vent 15, preventing the gases from flowing out of the vent 15 in the swivel connector 10. Should the gas flow abate, the flapper relaxes, opening the vent allowing the patient to breath in ambient air through the vent.

Note that, as will be shown, the axis of both the patient member 20 and the gas supply member 14 bend at approximately 45 degrees. By way of this bend, in one orientation of rotation, the bends counteract each other (e.g. subtract to zero degrees) and the axis of the patient member at the mask connection 21 is linear with the axis of the gas supply member 14 at the gas connection end 8. Similarly, by rotating the patient member 20 by 180 degrees with respect to the gas supply member 14, the bends work in tandem to form an elbow of approximately 90 degrees (e.g. add to 90 degrees). Although, in these examples, two 45 degree members 14/20 are shown, any angle is anticipated, including either equal or unequal angles. For example, two 30 degree angles result in an adjustment from zero degrees to 60 degrees, etc.

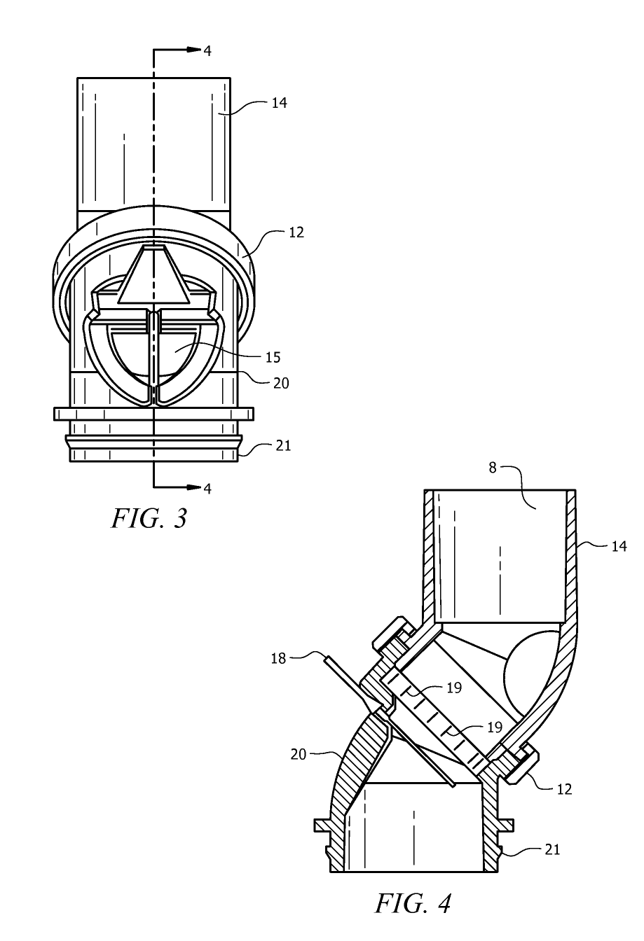

Referring to FIG. 3, an elevational view of the swivel connector 10 is shown. In this, the patient member 20 is connected to the gas supply member 14 by the collar 12, which in this example, fits over the gas supply member 14 and snaps onto the patient member 20. This is one example of how two members 14/20 are held together while permitting rotation of one member 14/20 with respect to the other member 20/14, at preferably 360 degrees of rotation. Any other form of connection is equally anticipated.

Referring to FIG. 4, a sectional view of the swivel connector 10 along lines 4-4 of FIG. 3 is shown. In this, multiple protrusions 19 on the gas supply member 14 are visible. These protrusions 19 interface with the detents 22 of the patient member, providing some resistance to rotation, helping to hold the gas supply member 14 in position with respect to the patient member 20 after positioned by a clinician twisting one member 14/20 with respect to the other member 20/14. Note, the optional protrusions 19 and detents 22 are anticipated to be on opposite members 14/20 and it is equally anticipated that any number of protrusions 19 and detents 22 are present, including one. For example, having ten detents 22 and one protrusion 19 allows for adjustment in 36 degree increments. Likewise having 10 detents and ten protrusions 19 also allows for adjustments in 36 degree increments.

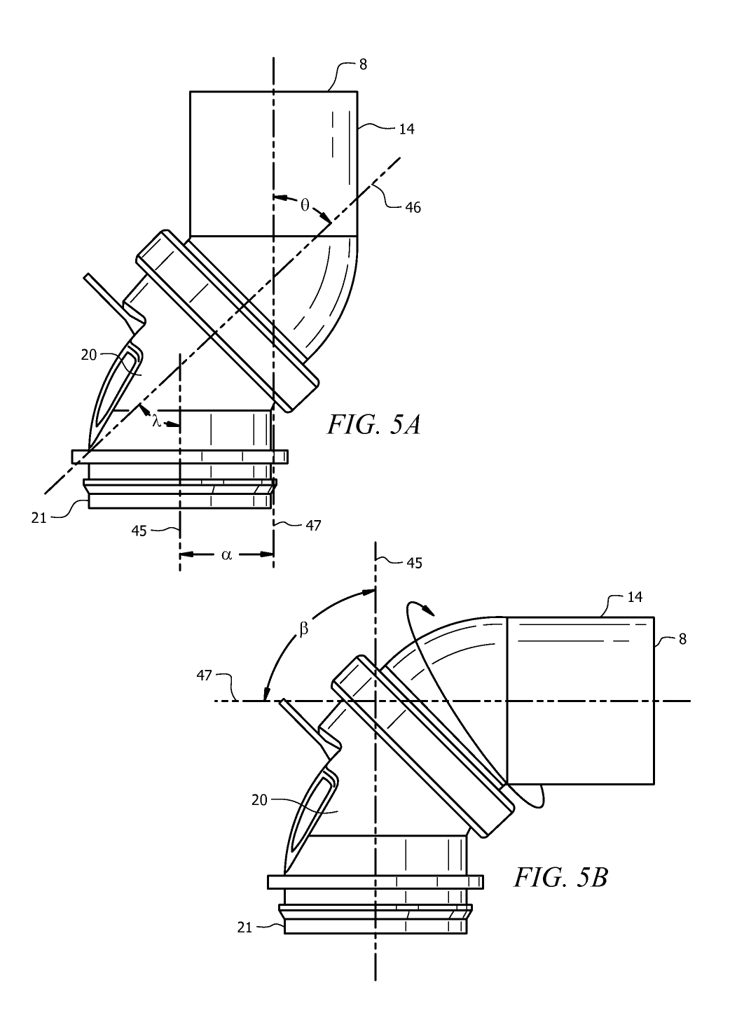

Referring to FIG. 5A and FIG. 5B, plan views of the swivel connector 10 in a straight configuration (FIG. 5A) and an elbow configuration (FIG. 5B) are shown.

The axis 45 at the first end of the patient member 20 (patient facemask connection) is offset from the axis 46 of both the second ends of the patient member 20 and gas supply member 14. This angle is denoted as λ and is preferably 45 degrees, though any angle from one degree to 89 degrees is anticipated. Likewise, the axis 47 at the first end of the gas supply member 14 (gas connection end 8) is offset from the axis 46 of both the second ends of the patient member 20 and gas supply member 14. This angle is denoted as θ and is preferably 45 degrees, though any angle from one degree to 89 degrees is anticipated. When λ and θ are both 45 degrees, through rotation of the patient member 20 with respect to the gas supply member 14, any angle of zero degrees (α) to 90 degrees (β) between axis 45 and 47 is possible. Note, although λ and θ are shown as the same 45 degree angles, there is no limitation of 45 degrees or that both λ and θ are the same angle. For example, in an alternate embodiment, λ is 40 degrees and θ is 30 degrees, etc.

In FIG. 5A, the axis 45 of the patient member 20 where the patient member 20 interfaces with the facemask 90 is substantially parallel (indicated by angle α, which is substantially zero) to the axis 47 of the gas supply member 14 where the gas supply member 14 interfaces to the gas supply tube 2. Therefore, the gas supply tube (not shown) is positioned to extend somewhat directly from the patient's face in the direction of the axis 45 and 47.

In FIG. 5B, the axis 45 of the patient member 20 where the patient member 20 interfaces with the facemask 90 is substantially at a right angle (90 degrees indicated by angle β) to the axis 47 of the gas supply member 14 where the gas supply member 14 interfaces to the gas supply tube 2 and, therefore, the gas supply tube (not shown) is positioned to extend sideways from the patient's face. For example, with the use of a nebulizer in the gas supply, the patient needs to be more or less vertical. If the patient is sitting up, the clinician twists the swivel connector 10 into the straight configuration as shown in FIG. 5A for effective use of the nebulizer.

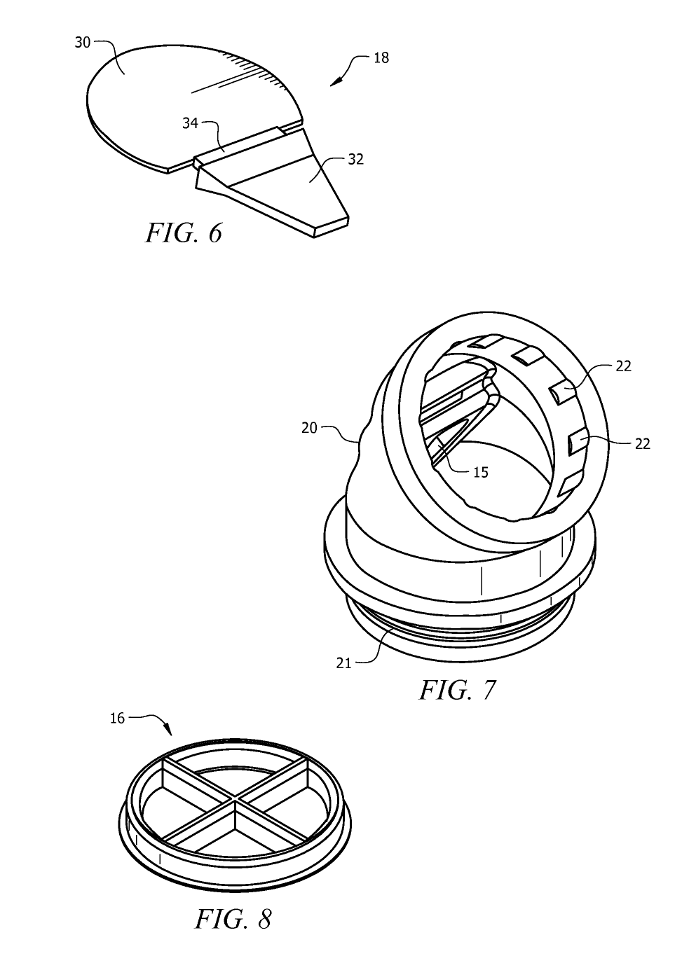

Referring to FIG. 6 through FIG. 10 perspective views of exemplary individual components of the swivel connector 10 are shown. FIG. 6 shows an exemplary flapper 18 of the flapper valve 16/18. The flapper 18 of the swivel connector 10 has a cover portion 30 that rests against the screen 16 when gases from the gas tube interface 8 are not flowing. This allows fluid communications between the patient member 20 and ambient air through the vent 15 should a failure occur, leading to lack of flow of the gases (e.g., a kink in the supply tube). When the gases from the gas tube interface 8 flow, the cover portion 30 bends at the inflection point 34 and occludes the vent 15 so that at least most of the gases flow to the patient rather than escaping out of the vent 15. A handle portion 32 is provided to hold and place the flapper 30 during manufacture. It is anticipated that the handle portion 32 is trimmed during manufacture after the flapper 18 is inserted. Note that other check valves 18 are fully anticipated providing the same safety feature should CPAP gases stop flowing.

In FIG. 7, the detents in the patient member 20 are shown. Although the patient member 20 is shown with a snap fit interface 21 for connecting to the facemask 90, any known facemask interface is anticipated.

In FIG. 8, an exemplary screen member 16 of the flapper valve 16/18 is shown. The cross bars of the screen member 16 hold the flapper 18 from flexing during exhalation of the patient.

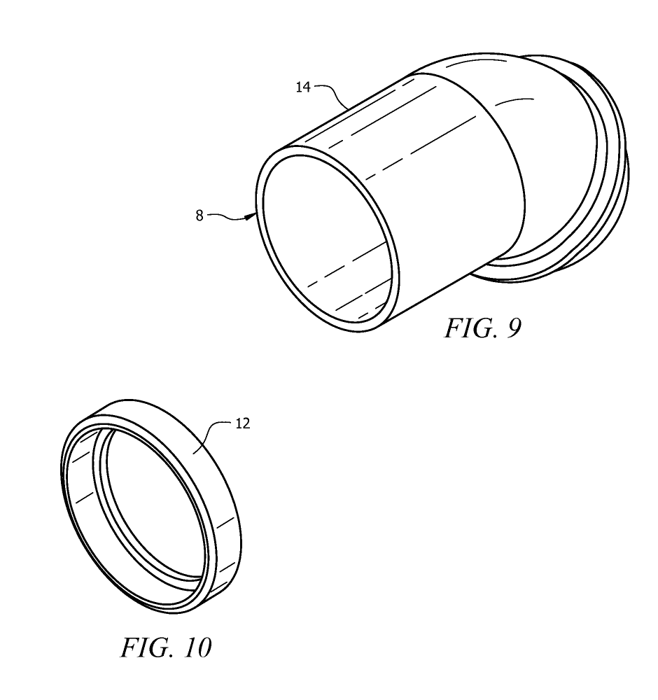

In FIG. 9, the gas tube interface 8 of the gas supply member 14 is shown.

In FIG. 10, an exemplary snap-on collar 12 is shown. Again, any mechanism for rotatably holding the gas supply member 14 to the patient member 20 is anticipated. This is one example of how two members 14/20 are held together while permitting rotation of one member 14/20 with respect to the other member 20/14, preferably with 360 degrees of rotation. Any form of connection is equally anticipated. It is preferred that this connection limit the amount of gases that are allowed to escape, though there is no requirement for a perfect seal. Additionally, through for example, a detent mechanism 19/22, the 360 degrees of rotation is divided into steps, holding one member 14/20 in position with respect to the other member 20/14 until external forces cause further rotation (e.g., forces provided by a clinician).

The swivel action of one member 14/20 with respect to the other member 20/14 from, for example, 90 degrees to straight does not require a change out of one component with another as was needed prior to the swivel connector 10. From a medical standpoint, comfort for the patient increases and usability for the clinician improves. From the comfort point of view the ability to swivel from one side to the other results in less pulling on the facemask 90 if the oxygen supply is only available from one side of the patient due to the, for example, a configuration of the ambulance or the size of the patient's hospital room. The clinician turns the swivel connector 10 into a straight configuration for breathing treatments (e.g., using a T-piece) to enable treatment without changing the mask, etc.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A swivel connector comprising:
a patient member, the patient member having a first end for connecting to a patient facemask, and the patient member having a second end, the first end of the patient member in fluid communication with the second end of the patient member; and
a gas supply member, the gas supply member having a first end for connecting to a supply of gases and the gas supply member having a second end that is rotatably coupled and fluidly coupled to the second end of the patient member, thereby the patient member is rotatable with respect to the gas supply member and gases flow between the patient member and the gas supply member;
wherein an axis of the first end of the patient member is at a non-zero angle with respect to an axis of the second end of the patient member and an axis of the first end of the gas supply member is at a non-zero angle with respect to an axis of the second end of the gas supply member and the axis of the second end of the patient member is in alignment with the axis of the second end of the gas supply member.

2. The swivel connector of claim 1, wherein the axis of the first end of the patient member is at a 45 degree angle with respect to the axis of the second end of the patient member and the axis of the first end of the gas supply member is at a 45 degree angle with respect to the axis of the second end of the gas supply member, thereby enabling the axis of the first end of the patient member to be adjusted to any angle between zero degrees and 90 degrees with respect to the axis of the first end of the gas supply member.

3. The swivel connector of claim 1, further comprising a vent, the vent formed in the patient member and in fluid communication with the first end and the second end of the patient member, the vent allowing exchange of gases between the first end of the patient member and outside of the patient member.

4. The swivel connector of claim 1, further comprising a check valve, the check valve closing when gases flow from the second end of the patient member to the first end of the patient member, thereby closing the vent and reducing exchange of gases between the first end of the patient member and outside of the patient member, and the check valve opening upon abatement of the gases flow, thereby allowing exchange of gases between the first end of the patient member and outside of the patient member.

5. The swivel connector of claim 4, wherein the check valve comprises a screen and a flapper, the screen positioned toward the first end of the gas supply member and the flapper abutting the screen on a side of the screen positioned toward the first end of the patient member and adjacent to the vent, whereas when the gases flow from the second end of the gas supply member, the flapper deflects away from the screen and covers the vent.

6. The swivel connector of claim 1, wherein an interface between the second end of the patient member and the second end of the gas supply member comprises a detent mechanism, the detent mechanism holding the second end of the patient member in rotational position with respect to the second end of the gas supply member until a twisting force is applied.

7. The swivel connector of claim 1, wherein the first end of the patient member is removably connected to the patient facemask.

8. The swivel connector of claim 1, wherein the first end of the gas supply member is removably connected to a tube, the tube being connected to the source of the gases.

9. A method of adjusting an angle of a connection between a supply of gases and a facemask, the method comprising:
connecting a first end of a patient member to a gas supply port of the facemask, the patient member having a second end, the first end of a patient member in fluid communication with the second end of a patient member;
connecting a first end of the gas supply member to the supply of gases, the gas supply member having a second end that is rotatably coupled and fluidly coupled to the second end of the patient member, thereby the patient member is rotatable with respect to the gas supply member and gases flow between the patient member and the gas supply member, and wherein the axis of the first end of the patient member is at a 45 degree angle with respect to the axis of the second end of the patient member and the axis of the first end of the gas supply member is at a 45 degree angle with respect to the axis of the second end of the gas supply member, thereby enabling the axis of the first end of the patient member to be adjusted to any angle between zero degrees and 90 degrees with respect to the axis of the first end of the gas supply member; and
rotating the patient member with respect to the gas supply member, therefore adjusting the angle between the axis of the first end of the patient member and the axis of the first end of the gas supply member from zero degrees to 90 degrees.

10. The method of claim 9, wherein the step of connecting the first end of a patient member to the gas supply port of the facemask includes snapping the first end of a patient member into the gas supply port of the facemask.

11. The method of claim 9, wherein the step of connecting the first end of the gas supply member to the supply of gases includes press fitting a tube onto the first end of the gas supply member, the tube carrying the gases from the supply of gases to the first end of the gas supply member.

12. The method of claim 9, wherein the step of rotating comprises:
turning one of the patient member and the gas supply member while holding still the other of the patient member and the gas supply member.

13. A system for providing gases from a tube to a patient, the system comprising:
a facemask for interfacing with an airway of the patient;
a patient member, the patient member having a first end connected to a port on the facemask and the patient member having a second end, the first end in fluid communication with the second end; and
a gas supply member, the gas supply member having a first end for connecting to the tube and the gas supply member having a second end that is rotatably coupled and fluidly coupled to the second end of the patient member, thereby the patient member is rotatable with respect to the gas supply member and gases flow between the patient member and the gas supply member;
wherein an axis of the first end of the patient member is at a non-zero angle with respect to an axis of the second end of the patient member and an axis of the first end of the gas supply member is at a non-zero angle with respect to an axis of the second end of the gas supply member and the axis of the second end of the patient member is in alignment with the axis of the second end of the gas supply member.

14. The system for providing gases from a tube to a patient of claim 13, wherein the axis of the first end of the patient member is at a 45 degree angle with respect to the axis of the second end of the patient member and the axis of the first end of the gas supply member is at a 45 degree angle with respect to the axis of the second end of the gas supply member, thereby enabling the axis of the first end of the patient member to be adjusted to any angle between zero degrees and 90 degrees with respect to the axis of the first end of the gas supply member.

15. The system for providing gases from a tube to a patient of claim 13, further comprising a check valve, the check valve closing upon flow of gasses from the first end of the gas supply member, thereby reducing flow of the gasses to ambient of the system for providing gases.

16. The system for providing gases from a tube to a patient of claim 15, further comprising a vent, the vent formed between the first end of the patient member and the check valve, the vent allowing flow of gases between the first end of the patient member and ambient air upon abatement of a flow of gases flowing into the first end of the gas supply member.

17. The system for providing gases from a tube to a patient of claim 16, wherein the check valve comprises a screen and a flapper, the screen positioned toward the first end of the gas supply member and the flapper abutting the screen on a side of the screen positioned toward the first end of the patient member whereas, the gases from the first end of the gas supply member push the flapper away from the screen and against the vent, sealing the vent and allowing the gases from the first end of the gas supply member to reach the first end of the patient member.

18. The system for providing gases from a tube to a patient of claim 13, wherein an interface between the second end of the patient member and the second end of the gas supply member comprises a detent mechanism, the detent mechanism holding the second end of the patient member in rotational position with respect to the second end of the gas supply member until a twisting force is applied.

19. The system for providing gases from a tube to a patient of claim 13, wherein the first end of the patient member is removably connected to the facemask.

20. The system for providing gases from a tube to a patient of claim 13, wherein the first end of the gas supply member is removably connected to a tube, the tube being connected to the source of the gases.
